(19) United States
(12) Patent Application Publication
(10) Pub. No.: US 2005/0058835 A1
(43) Pub. Date: Mar. 17, 2005

(54) Impregnation of polymeric substrates wit antimicrobal substances using superficial fluids

(76) Inventors: Steven Melvyn Howdle, Nottingham (GB); Roger Bayston, Nottingham (GB); Paul Brian Webb, Fife (GB)

Correspondence Address: BAKER & BOTTS, 30 ROCKEFELLER PLAZA, NEW YORK, NY, 10112, US

(21) Appl. No.: 10/496,425

(22) PCT Filed: Nov. 20, 2002
(86) PCT No.: PCT/GB02/05208

(30) Foreign Application Priority Data
Nov. 20, 2001 (GB) 0127786.2

(51) Int. Cl. A61L 2/00

(52) U.S. Cl. 428/411.1; 427/2.1

(57) ABSTRACT

A method of impregnating a polymeric substrate with an antimicrobial substance or precursor thereto, in which said substance is impregnated into said substrate as a solution, an emulsion or a suspension in a supercritical fluid. Additionally, there is provided a method of impregnating a substantially transparent polymeric substrate with an antimicrobial substance or precursor thereto, wherein the polymeric substrate is capable of being swelled by a swelling agent which contains dissolved, suspended or emulsified therein said antimicrobial substance or precursor thereto, so as to permit impregnation of the polymeric substrate with the antimicrobial substance or precursor thereto. There is also provided a device obtained by such methods.

This invention relates to an improved method for the impregnation of antimicrobial substances into implantable medical devices and to devices obtained by way of such a method.

The infection of implantable medical devices (especially partially-implanted devices) is a major concern in healthcare. In the case of central venous catheters (cvc), in the USA, the infection rate is cited as 16% with a direct mortality rate of 25%, usually from generalised sepsis.

Other device examples include wound drains, external ventricular drains and voice prostheses. The devices usually have to be removed in order to eradicate the infection, interrupting vital therapeutic programmes and causing distress and further risk to the patient.

The causative organisms of such infections comprise fungi (e.g. Candida species) and Staphylococci. Implantable devices are infected preferentially by microbes that are able to adhere to the material surface and proliferate in the form of biofilms or the like. Once established, it is known that these biofilm organisms are resistant to antibiotic therapy.

It is known that medical devices can be rendered antimicrobial by coating or impregnation with an antibiotic substance. A major disadvantage of this approach is that when exposed to flow conditions, such as in the vascular system, the antibiotic substance readily leaches from the implanted device into the surrounding environment e.g. into the blood of a patient. Further disadvantages include the implantable device becoming coated with a host-derived conditioning film consisting of glycoproteins and other substances, which inactivate the antimicrobial coating and if the antimicrobial coating is of a metal in elemental or salt form, said metal or salt becomes bound to host-derived proteins and subsequently inactivated. All these processes result in a rapid loss of antimicrobial protection of the device.

It has also been proposed to impregnate the device with small metal particles or other antimicrobial agent dissolved or suspended in an organic liquid. A disadvantage of this approach is that potentially toxic solvents may be retained in the medical device and may subsequently be released into the body of a patient. Furthermore, it is not always possible to swell the device material sufficiently to achieve desired impregnation. We have found that the foregoing disadvantages can be minimised by the use of a supercritical fluid as a carrier for the metal or the antimicrobial agent.

Accordingly, the present invention provides a method of impregnating polymeric substrate with an antimicrobial substance or precursor thereto, in which said antimicrobial substance is impregnated into said device as a solution, an emulsion or a suspension in a supercritical fluid.

The present invention also provides a polymeric substrate produced by the method described in the immediately-preceding paragraph.

In a second aspect of the present invention, there is provided a method of impregnating a substantially transparent polymeric substrate with an antimicrobial substance or precursor thereto, wherein the polymeric substrate is capable of being swelled by a swelling agent which contains dissolved, suspended or emulsified therein said antimicrobial substance or precursor thereto, so as to permit impregnation of the polymeric substrate with the antimicrobial substance or precursor thereto. In this particular aspect, the solvent need not be a supercritical fluid.

Preferably, the polymeric substrate is used in the manufacture of a medical device. More preferably, the medical device is an implantable medical device. The device may be totally or partially implanted.

The antimicrobial substance may be a precursor compound said precursor compound being readily decomposed in-situ to yield an active antimicrobial substance.

The precursor compound may be insoluble in the supercritical fluid and impregnated into a polymeric substrate as a suspension or emulsion in a supercritical fluid.

In a particularly preferred embodiment, the precursor is soluble in the supercritical fluid but the decomposition product is not. This enables an insoluble active anti-microbial substance to be effectively solubilised (in precursor form) so as to enable impregnation into a substrate. This is particularly important where it is not possible to swell or plastisize the device material sufficiently to enable impregnation with an insoluble material or where it is desired to build up domains of antimicrobial material within the polymer.

The medical device is preferably a partially implanted device. Alternatively the medical device may be a totally-implanted device.

The substrate and/or device is preferably manufactured, at least in part, from a polymeric, plastics or elastomeric material, for example polyacetals, polyamides, polyimides, polyesters, polycarbonates, polyurethanes, silicones, polyamide-imides, polyamide-esters, polyamide ethers, polycarbonate-esters, polyamide-ethers, polyacrylates; elastomers such as polybutadiene, copolymers of butadiene with one or more other monomers, butadiene-acrylonitrile rubber, styrene-butadiene rubber, polyisoprene, copolymers of isoprene with one or more other monomers, polyphosphazenes, natural rubber, blends of natural and synthetic rubber, polysiloxanes including polydimethylsiloxane and copolymers containing the diphenylsiloxane unit; polyalkylmethacrylates, particularly polymethylmethacrylate (PMMA), polyethylene, polypropylene, polystyrene, polyvinylacetate; polyvinylalcohol, and polyvinylchloride. Silicone polymers are particularly preferred.

The polymer may be a cross-linked polymer, for example polystyrene crosslinked with di-vinyl benzene (DVB). The implantable device may be made from an inorganic or inorganic-organic hybrid based polymer such as a silica aerogel or any other substance that can be penetrated by a supercritical fluid.

The medical device may, for example, be a central venous catheter, a wound drain, a voice prosthesis, a Continuous Ambulatory Peritoneal Dialysis (CAPD) device or a shunt to treat hydrocephalus or ascites or for haemodialysis.

“Antimicrobial substance”, as used herein, refers to essentially any antibiotic, antiseptic, disinfectant, etc., or combination thereof, effective for inhibiting the viability and/or proliferation of one or more microorganisms. Numerous classes of antibiotics are known and may be suitable for use in accordance with this invention. Such antibiotics may include, but are not necessarily limited to, tetracyclines (e.g., minocycline), rifamycins (e.g., rifampin), macrolides (e.g., erythromycin), penicillins (e.g., nafcillin), cephalosporins (e.g., cefazolin), other beta-lactam antibiotics (e.g., imipenem and aztreonam), aminoglycosides (e.g., gentamicin), chloramphenicol, sulfonamides (e.g., sulfamethoxyazole), glycopeptides (e.g., vancomycin), quinolones (e.g., ciprofloxacin), fusidic acid, trimethoprim, metronidazole, clindamycin, mupirocin, polyenes (e.g., amphotericin B), azotes (e.g., fluconazole), beta-lactam inhibitors, etc.

Examples of illustrative antibiotic substances that may be used in accordance with the present invention include minocycline, rifampin, erythromycin, nafcillin, cefazolin, imipenem, aztreonam, gentamycin, sulfamethoxazole, vanomycin, ciprofloxacin, trimethoprim, metronidazole, clindamycin, telcoplanin, mupirocin, azithromycin, clarithromycin, ofloxacin, lomefloxacin, norfloxacin, nalidixic acid, sparfloxacin, pefloxacin, amifloxacin, enoxacin, fleroxacin, ternafloxacin, tosufloxacin, clinafloxacin, sulbactarn, clavulanic acid, amphotericin B, fluconazole, itraconazole, ketoconazole, nystatin, and other like compounds.

Suitable antiseptics and disinfectants for use in this invention may include, for example, hexachlorophene, cationic bisiguanides (e.g., chlorohexidine, cyclohexidiene, etc.), iodine and iodophores (e.g., povidone-iodine), para-chloro-meta-xylenol, furan medical preparations (e.g., nitrofurantoin, nitrofurazone), methenamine, aldehydes (glutaraldehyde, formaldehyde, etc.), alcohols, and the like.

In a particularly preferred embodiment of the present invention, the antimicrobial substance comprises particles comprising or consisting of one or more metals for example, silver, zinc and/or copper. Alternatively, the antimicrobial element may comprise compounds, complexes or particles comprising or consisting of one or more metal salts, for example, silver oxide and/or copper oxide.

Generally, the particle size of the antimicrobial substance is between 10⁻⁹ m and 10⁻⁴ m, more preferably in the range between 10⁻⁹ m and 10⁻⁶ m, most preferably in the range between 10⁻⁹ m and 10⁻⁸ m. In particular, the particle size of the antimicrobial substance is preferably of the order of 5-200×10⁻⁹ m, most preferably 10-50×10⁻⁹ m.

Where a metal or metal complex is used as the antimicrobial substance, it is particularly preferred to build up domains of the substance within the polymer. These domains may be formed from one or more molecules of the impregnated substance or its decomposition product.

Preferably the substrate is impregnated with a soluble precursor of the antimicrobial substance. The soluble precursor may be a metal complex with a halogenated organic moiety. For example, a complex of silver with a fluorinated P-diketonate, in which the metal is surrounded by a fluorocarbon or hydrocarbon shell, may be used as the soluble precursor.

In a particularly preferred embodiment, the precursor to the antimicrobial substance is a metal complex. Particularly preferred ligands of the metal complex are fluorocarbons. Fluorocarbons are particularly effective CO₂-philes; a particularly preferred supercritical fluid in the present invention. The use of such encapsulating ligands in the design of the complex decreases their volatility, but enhances the solubility properties of the precursor complex by shielding the metal centre so that the supercritical CO₂ encounters only a hydrophobic shell. Particularly preferred metal complex precursors include Ag₂(hfpd)₂(COD)₂ where hfpd is 1,1,1,5,5,5-hexafluoro-2,4-pentanedione and COD is cyclo-octadiene and Ag(hfpd)L where L is either a multidentate amine, a multidentate glyme, or a phosphine or a thioether. In particular, Ag(hfpd) tetraamine [A] and Ag(hfpd) tetraglyme [B] are preferred as shown below.

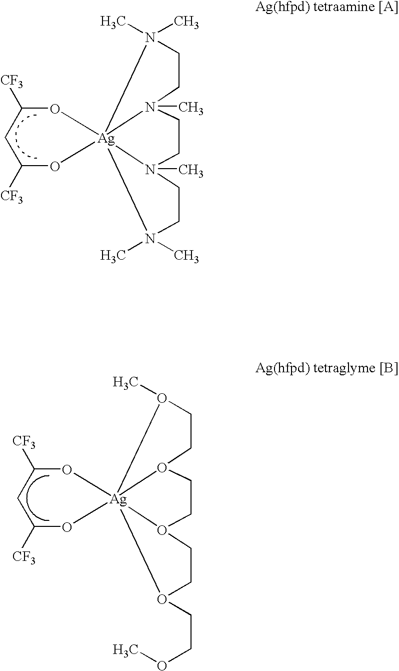

Preferably the soluble precursor decomposes upon exposure to external stimuli such as radiation (for example heat, light or ultra-violet radiation), electric current or chemical agent (for example hydrogen) to give the desired metal or metal oxide, together with chemical by-products of the decomposition reaction (free ligand residues). Most preferably, the precursor is reduced by any suitable reducing agent, most preferably hydrogen. An additional benefit of this process is that the metal particles may render the device radio-opaque.

In accordance with the present invention, two or more antimicrobial substances (e.g. silver and copper) may be impregnated into a single device. Preferably, each of the metals forms an individual precursor, leading to the deposition of individual particles in the device. Alternatively, the two metals may form alloyed particles, e.g. a silver/copper particle. A binuclear precursor may also be used containing two or more different types of metal.

The antimicrobial substance should preferably be mobile or be capable of being mobilised within the polymer matrix. In a particular preferred embodiment, the antimicrobial substance is capable of perfusing out of the polymeric substrate at a rate sufficient to maintain antimicrobial activity at the substrate surface. This is particularly important for in vivo systems where antimicrobial substances at the surface of a medical device are constantly washed away by physiological fluids, for example, blood, lymph, etc.

Where the antimicrobial substance is not, per se, capable of perfusion throughout the substrate, then it is preferably capable of being mobilised. For example, where the antimicrobial substance is a silver particle, the silver is capable of being solubilised as silver ions which can perfuse out of the substrate. For a silver particle with a sufficiently high surface area, such as the particle sizes discussed above, particularly nano particles, the silver is easily converted to silver ions at a rate sufficient to replenish silver ions washed from the surface of the substrate. It is also possible to apply an electric current to the substrate to increase or trigger the dissolution of the metal particles. This is particularly useful where one requires a boost in the antimicrobial activity or to mobilise antimicrobial substances that are impregnated deep within the substrate.

The supercritical fluid is preferably carbon dioxide (CO₂).

Alternatively, the supercritical fluid may be one of water, nitrogen, dinitrogen oxide, carbon disulphide, saturated or unsaturated aliphatic C₂₋₁₀ hydrocarbons, such as ethane, propane, butane, pentane, hexane, or ethylene, and halogenated derivatives thereof such as for example carbon tetrafluoride or tetrachloride, carbon monochloride trifluoride, and fluoroform or chloroform, C₆₋₁₀ aromatics such as benzene, toluene, or xylene, C₁₋₁₃ alcohols such as methanol, ethanol and isopropanol, sulphur halides such as sulphur hexafluoride, or ammonia, xenon, krypton or the like.

The supercritical fluid may also be used to extract conventional processing residue derived from, e.g. catheter production.

In accordance with the second aspect of the present invention, suitable swelling agents include hydrocarbon solvents such as hexane, benzene, xylene and toluene; ether type solvents such as diethyl ether, tetrahydrofuran, diphenyl ether, anisole and dimethoxybenzene;

halogenated hydrocarbon solvents such as methylene chloride, chloroform and chlorobenzene; ketone type solvents such as acetone, methyl ethyl ketone and methyl isobutyl ketone; alcohol type solvents such as methanol, ethanol, propanol, isopropanol, n-butyl alcohol and tert-butyl alcohol; nitrile type solvents such as acetonitrile, propionitrile and benzonitrile; ester type solvents such as ethyl acetate and butyl acetate; carbonate type solvents such as ethylene carbonate and propylene carbonate; and the like. These may be used singly or two or more of them may be used in admixture.

After impregnation has been effected, the swelling agent may be removed by any suitable method, for example, evaporation, washing, decomposition and the like. Low pressures may be used to extract solvent from the polymer substrate. In a particularly preferred embodiment, a supercritical fluid may be used to impregnate the polymeric material and/or remove swelling agent therefrom.

In a particularly preferred embodiment, the polymeric substrate of the second aspect of the present invention is used in the manufacture of a wound dressing. The substrate is preferably a block polymer or copolymer of the type described above. Most preferably the polymer is a silicone polymer. Preferably the wound dressing is in sheet form. The substantially transparent nature of the dressing is particularly important as it enables the wound to be observed without removing the dressing.

The term transparent is intended to mean that the polymer enables an observer to see clearly through a sheet constructed therefrom. Preferably, the sheet has in excess of 50% visible light transmission through a sheet of 2 mm thickness, more preferably greater than 70%, more preferably greater than 90%, most preferably greater than 95% light transmission.

Preferably, the polymer contains a u.v. blocker which substantially precludes u.v. transmission. This is particularly important for sensitive wounds such as burns. Such u.v blockers may be selected from 2-(2′-hydroxyphenyl) benzotriazoles, 2-hydroxybenzophenones, esters of substituted and unsubstituted benzoic acids, acrylates and oxalamides.

The wound dressing may be of any suitable shape. A particularly preferred embodiment is a substantially circular disc with an aperture at its centre or thereabouts, for encircling a tube, for example a catheter. The patch preferably has a broken side so the disc can be placed around a catheter or tube which is already in use. In practice, the patch resembles a flexible polo mint with a broken side.

The method according to the present invention may also be used to treat other plastics devices in non-medical areas, e.g. drain pipes, water supply pipes, air conditioning units or feed-production machinery.

The invention will now be illustrated, by way of the following examples and with reference to the single figure of the accompanying drawing.

### EXAMPLE 1

Cross-linked polystyrene beads (ca. 200 mg) were placed in a high pressure autoclave. An organometallic precursor, silver 1,1,1,5,5,5-hexafluoro-2,4-pentanedione (Ag(hfpd)L) (ca. 170 mg), where L was either (a) a multidentate amine (1,1,4,7,10,10-hexamethyltriethylene tetra-amine), (b) a multidentate glyme(tetraethylene glycol dimethyl 25 ether), (c) a phosphine or (d) a thioether) was added. The autoclave was sealed and filled with supercritical CO₂, to a pressure of 4000 psi and maintained at 40° C., to dissolve the organometallic precursor and to impregnate the precursor into the cross-linked polystyrene beads. The autoclave was then depressurised, filled with H₂ to a pressure of ca. 1000 psi and warmed to 60° C. The reduction with H₂ resulted in full decomposition of the metal co-ordination complex (Ag(hfpd)L) to yield nanometre-sized particles of silver metal. Following decomposition, the polystyrene beads were treated with supercritical CO₂ to remove any non-decomposed organometallic precursor or any unwanted by-products of the decomposition reaction. Samples of cross-linked polystyrene beads treated with each of the four precursors (a)-(d) were analysed by powder X-ray diffraction (XRD) which indicated the presence of metallic silver particles within the beads and the absence of any silver precursor complexes. Gravimetric analysis showed substantial increases in the mass of the cross-linked polystyrene beads following treatment by this process, also indicating that impregnation had taken place. Analysis of the treated beads by Transmission Electron Microscopy (TEM) confirmed that the beads contained small particles of metallic silver uniformly distributed within the polymer. The loading of the silver nanoparticles within the polymeric substrate was approximately 2% by weight.

### EXAMPLE 2

Cross-linked polystyrene beads (ca. 200 mg) were placed in a high pressure autoclave. An organometallic precursor, copper 1,1,1,5,5,5-hexafluoro-2,4-pentanedione (Cu(hfpd)L) (ca. 170 mg), where L was either (a) a multidentate amine, (b) a multidentate glyme, (c) a phosphine or (d) a thioether, was added. The organometallic precursor complexes were impregnated into the polymeric beads using supercritical CO₂ and decomposed using H₂ according to the method described in Example 1. Supercritical CO₂ was again used to remove any non-decomposed organometallic precursor and any unwanted by-products of the decomposition reaction.

### EXAMPLE 3

Cross-linked polystyrene beads (ca. 200 mg) were placed in a high pressure autoclave. An organometallic precursor, silver 1,1,1,5,5,5-hexafluoro-2,4-pentanedione (Ag(hfpd)L) (ca. 170 mg), where L was either (a) a multidentate amine or (b) a multidentate glyme was added. The precursor complexes were impregnated into the polymeric beads using supercritical CO₂ according to the method described in Example 1. The infused beads were then exposed to ultra-violet light, which caused the precursor complex to decompose, yielding nanometre-sized particles of silver within the polymer. Supercritical CO₂ was then used to remove any non-decomposed organometallic precursor and any unwanted by-products of the decomposition reaction.

Samples of polystyrene beads treated with each of the precursors (a) and (b) were analysed by powder X-ray diffraction (XRD) and gravimetric analysis, which confirmed the presence of silver nanoparticles within the polymeric substrate. TEM showed that the distribution of the nanoparticles was uniform and that the loading of the silver nanoparticles within the polymeric substrate was approximately 2% by weight.

### EXAMPLE 4

Ultra high molecular weight polyethylene (UHMWPE) was placed in a high pressure autoclave. An organometallic precursor, silver 1,1,1,5,5,5-hexafluoro-2,4-pentanedione (Ag(hfpd)L) (ca. 170 mg), where L was either (a) a multidentate amine or (b) a multidentate glyme was added. The precursor complexes were impregnated into the UHMWPE using supercritical CO₂ according to the method described in Example 1. The precursor complex was then decomposed using either hydrogen or ultra-violet light according to the methods described in the previous Examples. Supercritical CO₂ was used to remove any non-decomposed organometallic precursor and any unwanted by-products of the decomposition reaction.

### EXAMPLE 5

A silicone catheter was placed in a high pressure autoclave. An organometallic precursor, silver 1,1,1,5,5,5-hexafluoro-2,4-pentanedione (Ag(hfpd)L) (ca. 170 mg), where L was either (a) a multidentate amine or (b) a multidentate glyme, was added. The precursor complexes were impregnated into the catheter using supercritical CO₂ according to the method described in Example 1. The precursor complex was then decomposed using H₂. Supercritical CO₂ was then used to remove any non-decomposed organometallic precursor and any unwanted by-products of the decomposition reaction.

Samples of the catheters treated with each of the precursors (a) and (b) were analysed by powder x-ray diffraction (XRD) and gravimetric analysis, which confirmed the presence of silver nanoparticles within the polymeric silicone substrate. TEM showed that the distribution of the nanoparticles was uniform and that the loading of the silver nanoparticles within the polymeric substrate was approximately 2% by weight.

### EXAMPLE 6

A catheter was impregnated with silver particles using the method described in Example 5. This was tested for antimicrobial activity by the following method. A test bacterial strain (Staphylococcus epidermidis) isolated from an infected implant was incubated in tryptone soy broth (TSB, Oxoid Ltd, Basingstoke, UK) overnight at 37° C., and one drop of this was transferred to 10 mL of TSB and re-incubated for 3 hours at 37° C. with shaking. This early log phase culture was diluted 1/1000 in saline and used to inoculate an Isosensitest agar plate (Oxoid Ltd, Basingstoke, UK). Wells were cut approximately 5 mm apart using a special cutter and 8 mm segments of the impregnated catheter were placed so that their long axes were parallel to the long axis of the bridge between the two wells. This ensured that the cut edges of the catheter did not contact the agar. The plate was then incubated overnight at 37° C. and examined for zones of inhibition. The accompanying drawing is a photograph of the plate and clearly shows zones (1,2,3) of antimicrobial inhibition surrounding the catheter segments.

In a control experiment, a similar catheter was taken and treated with supercritical carbon dioxide in the absence of the metal precursor. Antimicrobial testing by the method described in the preceding paragraph showed no zones of antimicrobial inhibition around the device.

1. A method of impregnating an implantable medical device or material capable of being formed into an implantable medical device with an active antimicrobial substance comprising particles of one or more metals or salts thereof, the method comprising impregnating the device or material with a precursor compound that is capable of being decomposed in-situ in the device or material to yield said active antimicrobial substance, the precursor being impregnated into the device as a solution, an emulsion or a suspension in the supercritical fluid, and decomposing the precursor compound to produce said active antimicrobial substance.

2. A method according to claim 1, in which the metals are selected from silver, zinc, copper and mixtures thereof.

3. A method according to claim 1, in which the metal salts are selected from silver oxide and copper oxide.

4. A method as claimed in any one of claims 1 to 3, in which the size of the particles is between 10⁻⁹ m and 10⁻⁴ m, more preferably in the range between 10⁻⁹ m and 10⁻⁶ m, most preferably in the range between 10⁻⁹ m and 10⁻⁸ m.

5. A method as claimed in claim 4, in which the size of the particles is between 5×10⁻⁹ m and 200×10⁻⁹ m.

6. A method as claimed in any one of claims 1 to 5, in which the precursor compound is insoluble in the supercritical fluid and is impregnated into the polymeric substrate as a suspension or emulsion in a 30 supercritical fluid, or is soluble in the supercritical fluid and is impregnated into the polymeric substrate as a solution.

7. A method as claimed in any preceding claim, in which the device or material capable of being formed into the device is impregnated with a soluble precursor of the antimicrobial substance.

8. A method as claimed in claim 7, in which the soluble precursor is a metal complex with a halogenated organic moiety.

9. A method as claimed in claim 8, in which the complex is of silver with a fluorinated β-diketonate.

10. A method as claimed in claim 9, in which the metal complex precursor is Ag₂ (1,1,1,5,5,5-hexafluoro-2,4-pentanedione)₂ (cyclooctadiene)₂ or Ag (1,1,1,5,5,5-hexafluoro-2,4-pentanedione) L, wherein L is a multidentate amine, a multidentate glyme, a phosphine or a thioether.

11. A method as claimed in any one of claims 8 to 10, in which the soluble precursor decomposes upon exposure to an external stimulus to give the desired metal or metal oxide and free ligand residues.

12. A method as claimed in claim 11, in which the external stimulus comprises radiation.

13. A method as claimed in claim 11, in which the external stimulus is a chemical agent, preferably hydrogen.

14. A method as claimed in any one of claims 2 to 4 and claim 12, in which two or more active antimicrobial substances are impregnated into a single device or material capable of being formed into the device.

15. A method as claimed in claim 14, in which each active antimicrobial substance is formed from an individual precursor, leading to the deposition of individual particles of each active antimicrobial substance within the device or material.

16. A method as claimed in claim 14, in which the precursor compound decomposes to form alloyed particles that comprise two or more active antimicrobial substances.

17. A method as claimed in claim 16, in which the alloyed particles are silver/copper particles.

18. A method as claimed in any preceding claim, wherein the active antimicrobial substance or the precursor thereto forms nanoparticles within the implantable medical device or material capable of being formed into an implantable medical device.

19. A method as claimed in any one of claims 1, 6, or 18, in which the implantable medical device or material capable of being formed into an implantable medical device is selected from a polymeric, plastics or elastomeric material.

20. A method as claimed in claim 19, in which the polymeric, plastics or elastomeric material is selected from the group consisting of polyacetals, polyamides, polyimides, polyesters, polycarbonates, polyurethanes, silicones, polyamide-imides, polyamide-esters, polyamide-ethers, polycarbonate-esters, polyimide-ethers, polyacrylates; elastomers such as polybutadiene, copolymers of butadiene with one or more other monomers, butadiene-acrylonitrile rubber, styrene-butadiene rubber, polyisoprene, copolymers of isoprene with one or more other monomers, polyphosphazenes, natural rubber, blends of natural and synthetic rubber, polysiloxanes including polydimethylsiloxane and copolymers containing the diphenylsiloxane unit; polyalkylmethacrylates, particularly polymethylmethacrylate (PMMA), polyethylene, polypropylene, polystyrene, polyvinylacetate; polyvinylalcohol, and polyvinylchloride.

21. A method as claimed in claim 19 or claim 20, in which the polymeric, plastics or elastomeric material is a cross-linked polymer.

22. A method as claimed in any one of claims 1, 6, 18 or 19, in which the implantable medical device or material capable of being formed into an implantable medical device comprises an inorganic or inorganic-organic hybrid based polymer.

23. A method as claimed in any preceding claim, in which the implantable medical device is a central venous catheter, a wound drain, a voice prosthesis, a continuous ambulatory peritoneal dialysis (CAPD) device, a shunt to treat hydrocephalus or ascites or for haemodialysis.

24. A method as claimed in any one of the preceding claims, in which the supercritical fluid is carbon dioxide (CO₂).

25. A method as claimed in any one of claims 1 to 23, in which the supercritical fluid is water, nitrogen, dinitrogen oxide or carbon disulphide.

26. A method as claimed in any one of claims 1 to 23, in which the supercritical fluid is a saturated or unsaturated aliphatic C₂₋₁₀ hydrocarbon.

27. A method according to claim 26, in which the supercritical fluid is ethane, propane, butane, pentane, hexane or ethylene and halogenated derivatives thereof.

28. A method as claimed in any one of claims 1 to 23, in which the supercritical fluid is a C₆₋₁₀ aromatic hydrocarbon.

29. A method according to claim 28, in which the supercritical fluid is benzene, toluene or xylene.

30. A method as claimed in any one of claims 1 to 23, in which the supercritical fluid is a sulphur halide, ammonia, xenon or krypton.

31. A method as claimed in any one of claims 1, 6, and 24 to 30, in which the supercritical fluid is used to extract conventional processing residue derived from the production of the implantable medical device or material.

32. A method substantially as described herein with reference to the examples.

33. A method of impregnating polymeric substrate of a substantially transparent implantable medical device or material capable of being formed into a substantially transparent implantable medical device with an antimicrobial substance comprising particles of one or more metals or salts thereof, the method comprising swelling the polymeric substrate of the implantable medical device or material capable of being formed into an implantable medical device with a swelling agent which contains dissolved, suspended or emulsified therein a precursor compound to said antimicrobial substance, so as to impregnate the polymeric substrate with the active antimicrobial substance precursor compound, and producing said antimicrobial substance in situo in said polymeric substrate from the precursor compound.

34. A method according to claim 33, wherein the swelling agent is selected from the group consisting of hydrocarbon solvents such as hexane, benzene, xylene and toluene; ether type solvents such as diethyl ether, tetrahydrofuran, diphenyl ether, anisole and dimethoxybenzene; halogenated hydrocarbon solvents such as methylene chloride, chloroform and chlorobenzene; ketone type solvents such as acetone, methyl ethyl ketone and methyl isobutyl ketone; alcohol type solvents such as methanol, ethanol, propanol, isopropanol, n-butyl alcohol and tert-butyl alcohol; nitrile type solvents such as acetonitrile, propionitrile and benzonitrile; ester type solvents such as ethyl acetate and butyl acetate; carbonate type solvents such as ethylene carbonate and propylene carbonate and mixtures thereof.

35. A implantable medical device or material capable of being formed into an implantable medical device obtained by the method of any one of the preceding claims.

36. A wound dressing obtained by a method according to claim 33 or 34.

37. A method of killing microbes by exposing microbes to an implantable medical device or material capable of being formed into an implantable medical device according to claim 35 or 36.

38. A method of producing a polymeric impregnated with an active antimicrobial substance comprising particles of one or more metals or salts thereof, the method comprising impregnating the polymeric substrate with a precursor compound that is capable of being decomposed in-situ to yield said active antimicrobial substance, said precursor compound being impregnated into said substrate as a solution, an emulsion or a suspension in a supercritical fluid, and producing said antimicrobial substance particles in situo in said substrate from said precursor compound.

39. A medical device or bulk plastics material capable of being formed into a medical device having impregnated in a body of polymeric, plastics, or elastomeric substrate particles of one or more metals or salts thereof adapted to produce metal ions having antimicrobial activity, the polymeric substrate permitting migration of the metal ions to a surface of the body of polymeric material at a rate sufficient to provide antimicrobial activity.

40. A device or material according to claim 39 in which the size of the particles is between 10⁻⁹ m and 10⁻⁴ m, more preferably in the range between 10⁻⁹ m and 10⁻⁶ m, most preferably in the range between 10⁻⁹ m and 10⁻⁸ m.

41. A device or material of claim 40 in which the particles comprise nanoparticles.

42. A device or material according to any one of claims 39 to 41 in which the particles comprise at least one of silver, zinc, copper, or salts thereof, or mixtures of the silver, zinc and/or copper or salts thereof.
